(19) United States
(12) Patent Application Publication
(10) Pub. No.: US 2008/0167612 A1
(43) Pub. Date: Jul. 10, 2008

(54) Intravenous catheter introducing device

(76) Inventors: Ming-Jeng Shue, Taichung City (TW); Deborah Huang, Taichung City (TW); Phillip Shue, Taichung City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/904,502

(22) Filed: Sep. 26, 2007

(30) Foreign Application Priority Data
Jan. 5, 2007 (TW) 096100447

(51) Int. Cl. A61M 5/158

(52) U.S. Cl. 604/110; 604/166.01; 604/272

(57) ABSTRACT

An intravenous catheter introducing device includes a needle hub received in a barrel and holding a needle cannula. The barrel has a slotted path with starting and ending regions, and a docking port communicated with the starting region to form a docking shoulder. An engaging peg is disposed on the needle hub, and is anchored on the docking shoulder in use. The peg is slidable along the slotted path to the ending region for drawing the needle cannula into the barrel by means of a spring. A receptacle frame is disposed on the barrel, and defines a guide way for guiding movement of a guided body of an actuating unit such that a pushing body shoves the peg from the docking shoulder to the starting region. A stabilizing unit is disposed to keep the peg moving with the guided body.

### CROSS-REFERENCES TO RELATED APPLICATIONS

This application claims priority to Taiwanese Application No. 096100447, filed Jan. 5, 2007, the disclosure of which is herein incorporated by reference.

### FIELD OF THE INVENTION

This invention relates to an intravenous catheter introducing device, more particularly to an intravenous catheter introducing device with a needle cannula which is retractable into a barrel for safe disposal.

### DESCRIPTION OF THE RELATED ART

Conventional medical devices or syringes for medicine injection, drawing blood samples, etc., have to be disposed of safely after use to avoid accidental needle pricks or undesirable contamination. Although a tip protector is provided to be sleeved on the device after use to ensure that the needle is covered, the user is exposed to the risk of being pricked by the needle when sleeving the tip protector on the used device. Therefore, there are available medical devices or syringes with a retractable needle that is retractable into a barrel or a plunger after the injection operation is completed, such as those disclosed in U.S. patent application Ser. Nos. 11/488,406 and 11/488,424 filed by the applicants, and U.S. Pat. Nos. 7,044,935, 7,204,813, and 7,211,064 issued to the applicants. However, further improvements are desirable in order to ensure easy and safe retraction of the needle and to simplify the construction of medical devices or syringes.

### SUMMARY OF THE INVENTION

The object of the present invention is to provide an intravenous catheter introducing device which has a simplified construction and which can be operated easily and safely to retract a used needle cannula.

According to this invention, the intravenous catheter introducing device includes a barrel, a needle cannula, a needle hub, a biasing member, an engaging peg, a receptacle frame, an actuating unit, and a stabilizing unit.

The barrel has front and rear open ends opposite to each other in a longitudinal direction, and a surrounding barrel wall which interconnects the front and rear open ends and which includes front and rear wall portions. The rear wall portion has an inner barrel wall surface which defines a passage, and which has a slotted path that extends through an outer barrel wall surface of the surrounding barrel wall, and that extends along the longitudinal direction to have starting and ending regions. The inner barrel wall surface further has a docking port which extends through the outer barrel wall surface, and which extends in a transverse direction to be communicated with the starting region so as to form a docking shoulder that faces towards the front open end.

The needle cannula has a front segment terminating at a tip end, and a rear connecting end opposite to the front segment.

The needle hub includes a front holding portion which is surrounded by the front wall portion and which holds the rear connecting end of the needle cannula, and a rear shell portion which is inserted into the passage such that the needle hub is slidable relative to the surrounding barrel wall along the axis between a position of use, where the front segment of the needle cannula extends forwardly of the front open end, and a disposal position, where the front segment of the needle cannula retreats into the passage. Preferably, the needle hub further includes an intermediate viewing-window portion between the front holding portion and the rear shell portion.

The biasing member is interposed between the rear shell portion and the inner barrel wall surface to bias the needle hub towards the disposal position.

The engaging peg is configured to be anchored on the docking shoulder against the biasing action of the biasing member in the position of use, and extends from the rear shell portion radially so as to be externally manipulated to move from the docking shoulder to the starting region. The engaging peg is dimensioned to be slidable along the slotted path to the ending region so as to move the needle hub to the disposal position.

The receptacle frame is disposed on the outer barrel wall surface, and includes front and rear guiding grooves which are spaced apart from each other in the longitudinal direction, and which cooperate with each other to define a guideway that extends over the docking port and the starting region in the transverse direction.

The actuating unit includes a guided body and a pushing body. The guided body has first and second guided sides that are guided respectively in the front and rear guiding grooves when the actuating unit is moved along the guideway between normal and actuating positions. The pushing body is configured such that when the guided body is in the normal position, the pushing body is distant from the engaging peg, and when the guided body is in the actuating position, the pushing body shoves the engaging peg to the starting region.

The stabilizing unit is disposed between the guided body and the engaging peg, and is configured such that the engaging peg is kept moving with the guided body until the engaging peg reaches the starting region, thereby ensuring steady movement of the engaging peg between the docking shoulder and the starting region.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is an exploded sectional view of a first preferred embodiment of an intravenous catheter introducing device according to this invention;

FIG. 2 is a fragmentary exploded perspective view of a portion of the first preferred embodiment;

FIG. 3 is a sectional view of the first preferred embodiment in a position of use;

FIG. 4 is a fragmentary partly sectioned view showing an actuating unit of the first preferred embodiment in a normal position;

FIG. 5 is a fragmentary partly sectioned view showing the actuating unit in an actuating position of the first preferred embodiment;

FIG. 6 is a sectional view of the first preferred embodiment in a disposal position;

FIG. 7 is a fragmentary partly sectioned view showing a safety guard mounted in the first preferred embodiment;

FIG. 8 is a fragmentary exploded perspective view of a portion of the second preferred embodiment of an intravenous catheter introducing device according to this invention;

FIG. 9 is a sectional view of the second preferred embodiment in a position of use;

FIG. 10 is a fragmentary partly sectioned view of an actuating unit in a normal position of the second preferred embodiment;

FIG. 11 is a schematic partly sectioned view of the second preferred embodiment in a disposal position;

FIG. 12 is a sectional view of the second preferred embodiment in the disposal position viewed from another angle;

FIG. 13 is a sectional view of a portion of the third preferred embodiment of an intravenous catheter introducing device according to this invention in a position of use;

FIG. 14 is a schematic partly sectioned view of the third preferred embodiment in a disposal position;

FIG. 15 is a sectional view of the third preferred embodiment in the disposal position;

FIG. 16 is a fragmentary exploded perspective view of the fourth preferred embodiment of an intravenous catheter introducing device according to this invention;

FIG. 17 is a sectional view of the fourth preferred embodiment in a position of use;

FIG. 18 is a fragmentary partly sectioned view of the fourth preferred embodiment showing an actuating unit in a normal position;

FIG. 19 is a schematic partly sectioned view of the fourth preferred embodiment in a disposal position; and

FIG. 20 is a sectional view of the fourth preferred embodiment in the disposal position.

### DETAILED DESCRIPTION OF THE PREFERRED EMBODIMENTS

Before the present invention is described in greater detail, it should be noted that same reference numerals have been used to denote like elements throughout the specification.

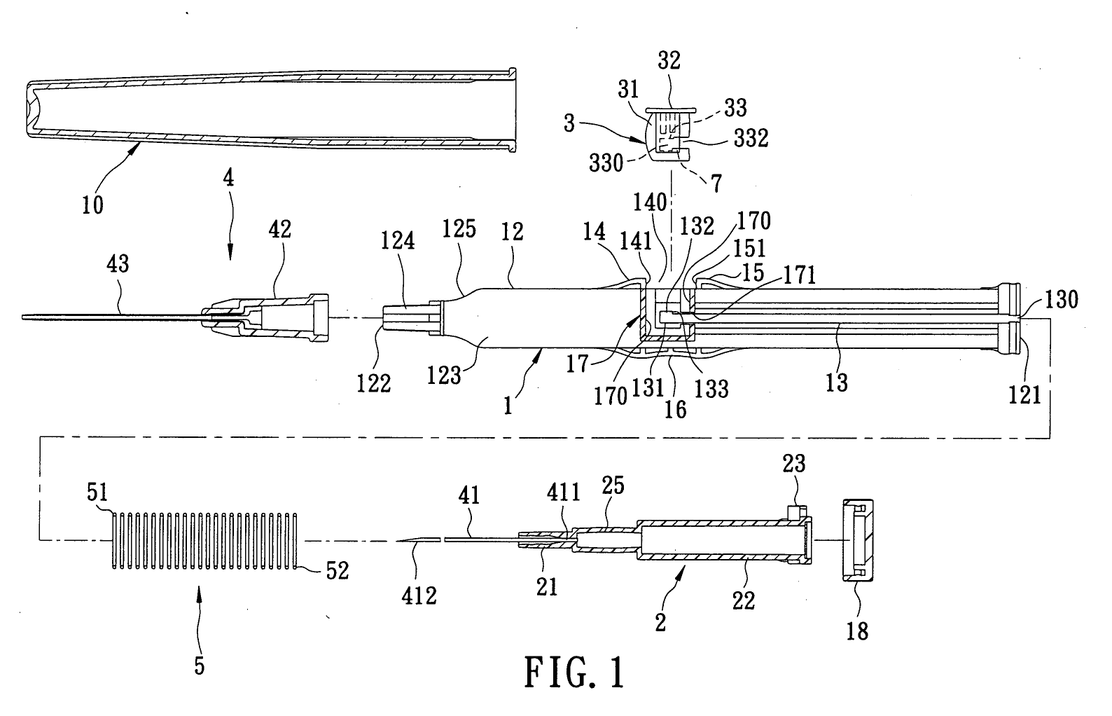

Referring to FIGS. 1 to 3, the first preferred embodiment of an intravenous catheter introducing device according to the present invention is shown to comprise a barrel 1, a needle cannula 41, a needle hub 2, a catheter connection assembly 4, a tip protector 10, a biasing member 5, an engaging peg 23, a receptacle frame 17, an actuating unit 3, and stabilizing unit 7.

The barrel 1 has front and rear open ends 122,121 opposite to each other in a longitudinal direction, and a surrounding barrel wall 12 which interconnects the front and rear open ends 122,121. The surrounding barrel wall 12 includes a small-diameter front wall portion 124 and a large-diameter rear wall portion 123 which are respectively disposed proximate to the front and rear open ends 122,121 to form a shoulder 125 therebetween. The rear wall portion 123 has an inner barrel wall surface which surrounds an axis in the longitudinal direction and which defines a passage 11, and an outer barrel wall surface opposite to the inner barrel wall surface in radial directions relative to the axis. The inner barrel wall surface has a slotted path 13 which extends through the outer barrel wall surface, and which extends along the longitudinal direction to have starting and ending regions 131,130 that are opposite to each other, and that are respectively distal from and proximate to the rear open end 121. The inner barrel wall surface further has a docking port 132 which extends through the outer barrel wall surface, and which extends in a first transverse direction to be communicated with the starting region 131 so as to form a docking shoulder 133 that faces towards the front open end 122. The inner barrel wall surface further has a plurality of ribs 126 formed at the shoulder 125. An end cap 18 is disposed to cover the rear open end 121. Preferably, the ending region 130 of the slotted path 13 is in a constricted form and extends into the end cap 18.

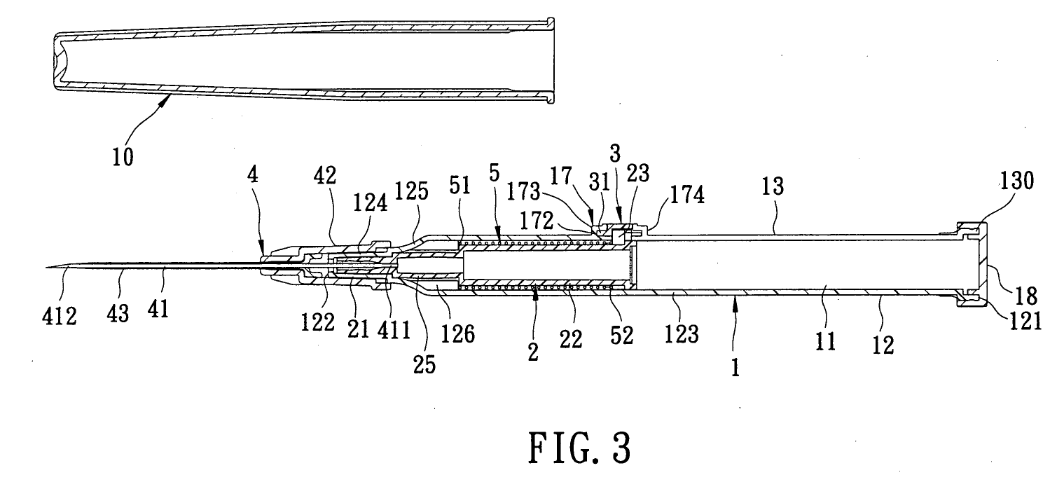

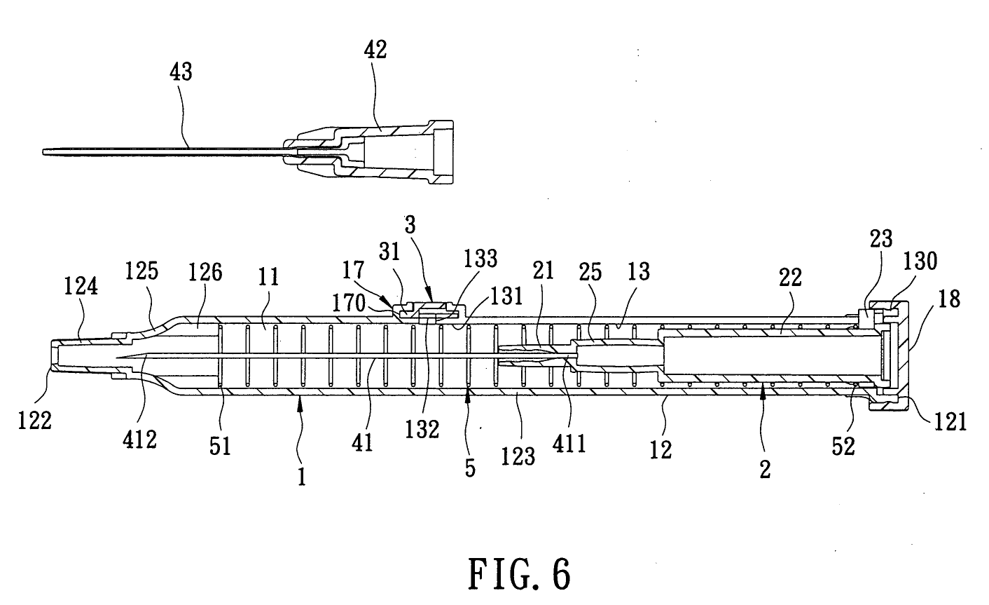

The needle cannula 41 has a front segment terminating at a tip end 412, and a rear connecting end 411 opposite to the front segment along the axis. The needle hub 2 includes a front holding portion 21 which is surrounded by the front wall portion 124 and which holds the rear connecting end 411 of the needle cannula 41, a rear shell portion 22 which is disposed opposite to the front holding portion 21 along the axis to be inserted into the passage 11, and an intermediate viewing-window portion 25 which is interposed between the front holding portion 21 and the rear shell portion 22 for permitting viewing of blood flowing therein. Thus, the needle hub 2 is slidable relative to the surrounding barrel wall 12 along the axis between a position of use, where the front segment of the needle cannula 41 extends forwardly of the front open end 122, as shown in FIG. 3, and a disposal position, where the front segment of the needle cannula 41 retreats into the passage 11, as shown in FIG. 6.

The catheter connection assembly 4 includes a catheter hub 42 and a flexible tubular catheter 43.

The tip protector 10 is removably sleeved on the barrel wall 12 for shielding the needle cannula 41.

The biasing member 5 is a coil spring which is disposed in the rear wall portion 123, and which has a front biasing end 51 that engages the ribs 126, and a rear biasing end 52 that engages the rear shell portion 22 such that the biasing member 5 is in a compressed state when the needle hub 2 is in the position of use so as to bias the needle hub 2 towards the disposal position. Hence, the biasing member 5 is disposed to surround the rear shell portion 22, and will not conceal the intermediate viewing-window portion 25 to obstruct viewing of flashback blood flow.

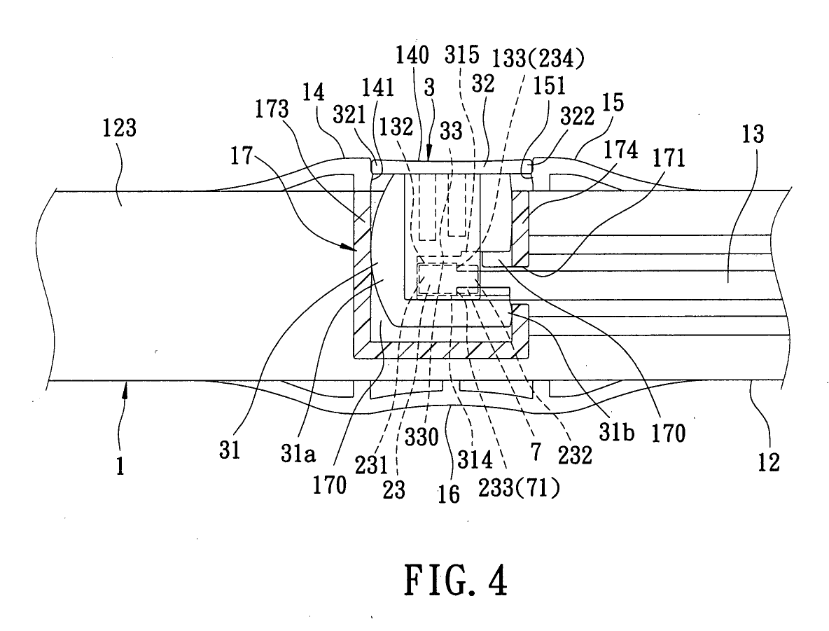

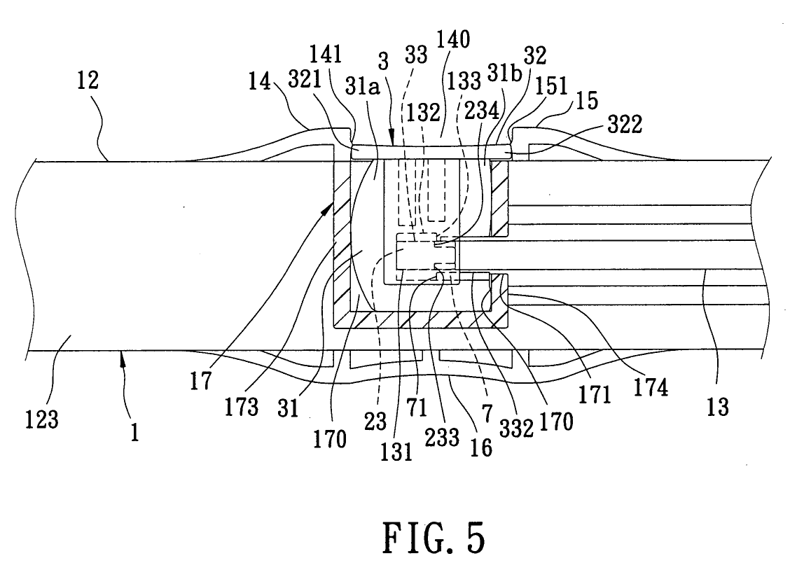

The engaging peg 23 is integrally formed with and extends radially from the rear shell portion 22. As shown in FIG. 4, in this embodiment, the engaging peg 23 has an enlarged head portion 231 and a tail portion 232 opposite to each other in the longitudinal direction, and left and right anchoring abutments 233,234 which are interposed between the enlarged head portion 231 and the tail portion 232. The right anchoring abutment 234 is anchored on the docking shoulder 133 when the needle hub 2 is in the position of use against the biasing action of the biasing member 5. The enlarged head portion 231 has a stud end which is disposed outwardly of the docking port 132 so as to be externally manipulated to move from the docking shoulder 133 to the starting region 131 of the slotted path 13, as shown in FIG. 5. The engaging peg 23 is slidable along the slotted path 13 to the ending region 130 so as to move the needle hub 2 to the disposal position, as shown in FIG. 6.

The receptacle frame 17 is integrally formed with and is disposed on the outer barrel wall surface of the barrel 1, and includes front and rear guiding grooves 170 which are spaced apart from each other in the longitudinal direction, and which cooperate with each other to define a guideway 172 that extends over the docking port 132 and the starting region 131 in the first transverse direction. The front and rear guiding grooves 170 respectively extend towards the front and rear open ends 122,121 to terminate at front and rear guiding walls 173,174, respectively. The rear guiding wall 174 is interrupted by the slotted path 13 at an opening 171 thereof so as to permit the engaging peg 23 to pass through the opening 171 when the needle hub 2 is moved from the position of use to the disposal position.

The receptacle frame 17 includes a receiving groove 175 which is disposed leftwardly of the slotted path 13, and which extends in the longitudinal direction to communicate the front guiding groove 170 with the rear guiding groove 170. The receiving groove 175 extends away from the slotted path 13 in a second transverse direction transverse to both the longitudinal direction and the first transverse direction to terminate at a shielding wall 176. The shielding wall 176 extends rightwards to terminate at a lateral edge 177.

The receptacle frame 17 further includes front and rear jamb walls 14,15 which are disposed rightwardly of the front and rear guiding grooves 170, respectively, and which are spaced apart from each other in the longitudinal direction to define a pushing path 140. The receptacle frame 17 further includes front and rear tiny projections 141,151 which are disposed respectively on the front and rear jamb walls 14,15. A lateral holding member 16 is disposed on the outer barrel wall surface of the barrel 1 and diametrically opposite to the pushing path 140 in the second transverse direction.

The actuating unit 3 includes a guided body 31, a pushing body 33, and a faceplate 32.

The guided body 31 has a major wall which extends in the longitudinal direction to terminate at first and second guided sides (31a,31b), and which has outer and inner major surfaces 311,312 opposite to each other in the second transverse direction. The first and second guided sides (31a,31b) are guided respectively in the front and rear guiding grooves 170 when the actuating unit 3 is moved along the guideway 172. In this embodiment, the inner major surface 312 has a cavity 330 which extends towards the outer major surface 311 to terminate at a ceiling surface 313, and which extends in the first transverse direction to terminate at left and right wall surfaces 314,315 that accommodate the engaging peg 23 therebetween when the guided body 31 is guided along the guideway 172. The left and right wall surfaces 314,315 extend rearwardly to form in the second guided side 31b a port 332. As shown in FIG. 5, the port 332 is registered with the slotted path 13 in the actuating position, thereby permitting the engaging peg 23 to be moved to the ending region 130.

In this embodiment, the pushing body 33 is integrally formed with and is disposed on the right wall surface 315. Thus, when the actuating member 3 is in a normal position, as shown in FIG. 4, the pushing body 33 is distant from the engaging peg 23, and the engaging peg 23 is engaged with the docking shoulder 133. When the actuating member 3 is in an actuating position, as shown in FIG. 5, the pushing body 33 shoves the engaging peg 23 to cause the latter to disengage from the docking shoulder 133 and to reach the starting region 131.

In this embodiment, the faceplate 32 is disposed on the guided body 31 distal from the cavity 330 in the first transverse direction, and extends in the longitudinal direction to have front and rear fitted edges 321,322. The pushing path 140 is of such a dimension as to be fittingly engaged with the front and rear fitted edges 321,322 of the faceplate 32.

In this embodiment, the stabilizing unit 7 is integrally formed with and is disposed on the left wall surface 314, and is formed with an auxiliary shoulder 71. Thus, referring to FIG. 4, in the normal position, the left anchoring abutment 233 of the engaging peg 23 abuts against the auxiliary shoulder 71.

In use, as shown in FIGS. 3 and 4, the tip end 412 of the needle cannula 41 is inserted into the patient's vein so as to introduce the tubular catheter 43 into the vein. Blood flowing into the intermediate viewing-window portion 25 of the needle hub 2 is visible so that the user can check whether the needle cannula 41 has been inserted properly into the vein to introduce the tubular catheter 43. The user can then separate the catheter hub 42 from the barrel 1 with one finger of the hand holding the barrel 1. Subsequently, the user can press the vein and the tubular catheter 43 with the other hand to complete the IV catheter introducing procedure.

As shown in FIGS. 5 and 6, the user pushes the faceplate 32 with the finger of the hand holding the barrel 1 along the pushing path 140 to reach the actuating position such that the front and rear fitted edges 321,322 of the faceplate 32 are brought into a press-fit with the front and rear jamb walls 14,15 by slipping over the front and rear tiny projections 141,151. At this time, the auxiliary shoulder 71 is caused to displace along with the guided body 31 to disengage from the left anchoring abutment 233 of the engaging peg 23. In addition, the pushing body 33 shoves the engaging peg 23 to the starting region 131. Consequently, the engaging peg 23 is permitted to move along the slotted path 13 by virtue of the biasing action of the biasing member 5 so as to be retained in the constricted ending region 130, such that the needle hub 2 is displaced to the disposal position and the used needle cannula 41 is retracted into the passage 11.

As illustrated, since the used needle cannula 41 can be disposed in the passage 11 immediately after completion of the IV catheter introducing procedure in a convenient and easy manner, accidental needle pricking can be prevented. Moreover, once the faceplate 32 is pushed, the front and rear fitted edges 321,322 are moved over the front and rear tiny projections 141,151 to be in a press-fit with the front and rear jamb walls 14,15. Thus, the faceplate 32 is retained in the actuated position to prevent removal of the actuating unit 3 from the pushing path 140.

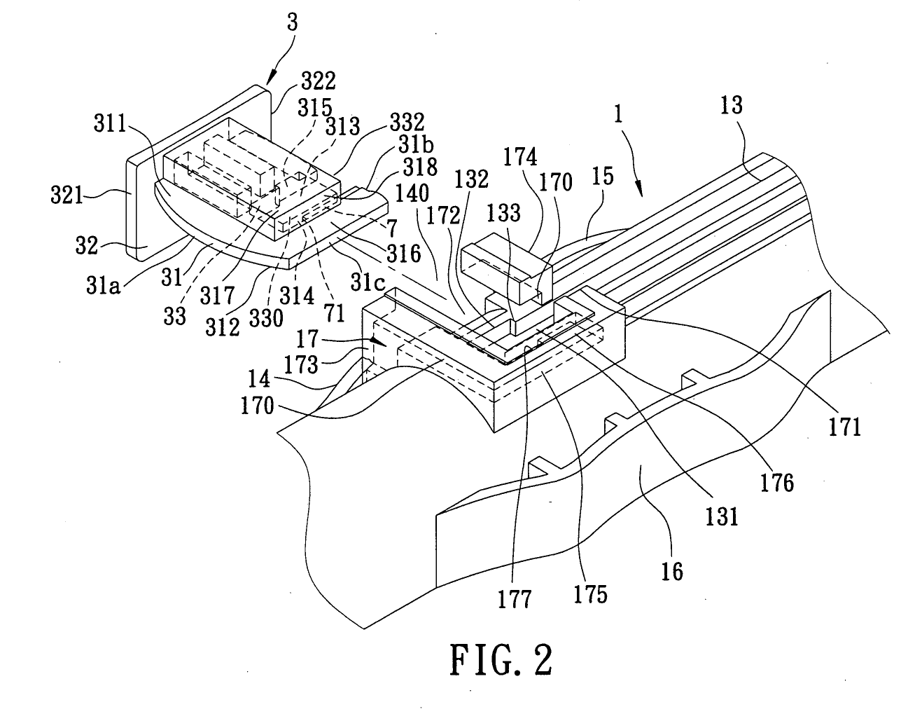

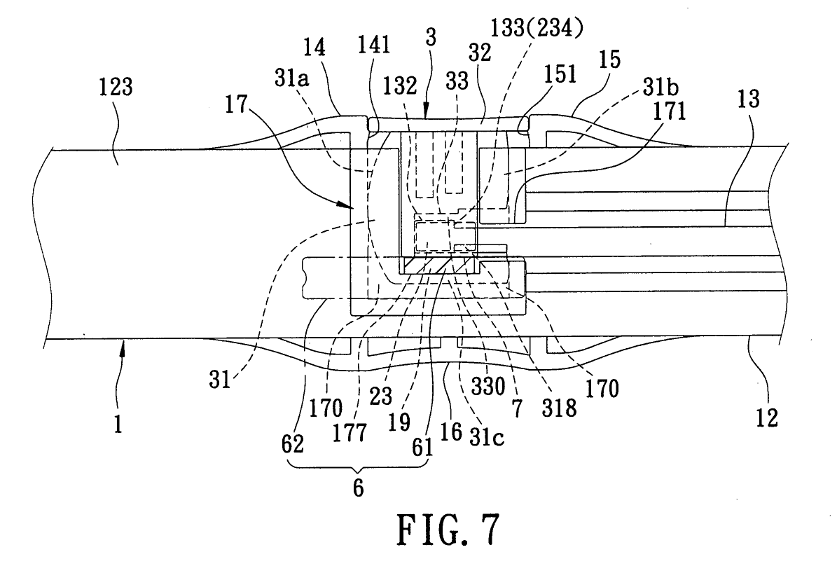

As shown in FIGS. 2 and 7, the major wall of the guided body 31 has a leading edge (31c) which joins the front and rear guided sides (31a,31b), and which is to be received in the receiving groove 175. The outer major surface 311 of the guided body 31 has lower and elevated regions 316,317 respectively proximate to and distal from the leading edge (31c). The elevated region 317 is raised to accommodate the contour of the ceiling surface 313 so as to form a head-on surface 318 which extends in the second transverse direction between the lower and elevated regions 316,317, and which is spaced apart from the lateral edge 177 by a clearance 19 when the actuating unit 3 is in the normal position. In order to prevent undesired movement of the actuating unit 3 during operation and carrying of the intravenous catheter introducing device, a safety guard 6 is provided, and has an insert portion 61 which is configured to be fittingly received in the clearance 19 so as to guard against inadvertent movement of the guided body 31 away from the normal position, and a head portion 62 which is disposed outwardly of the barrel 1 so as to permit detachment of the insert portion 61 from the clearance 19.

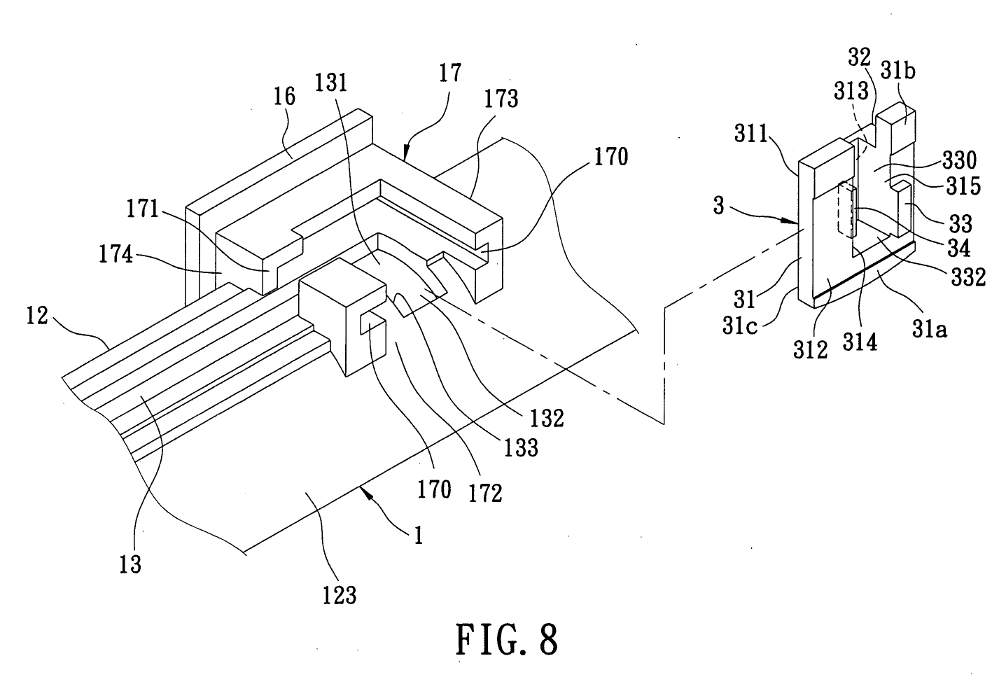

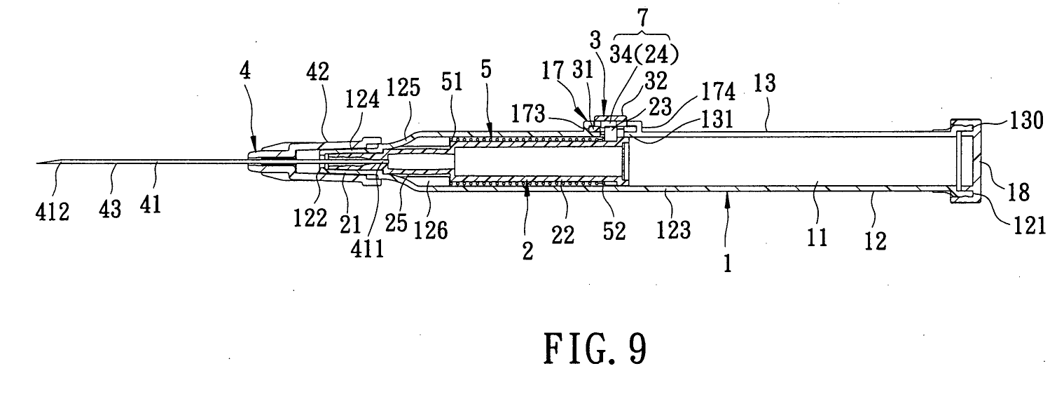

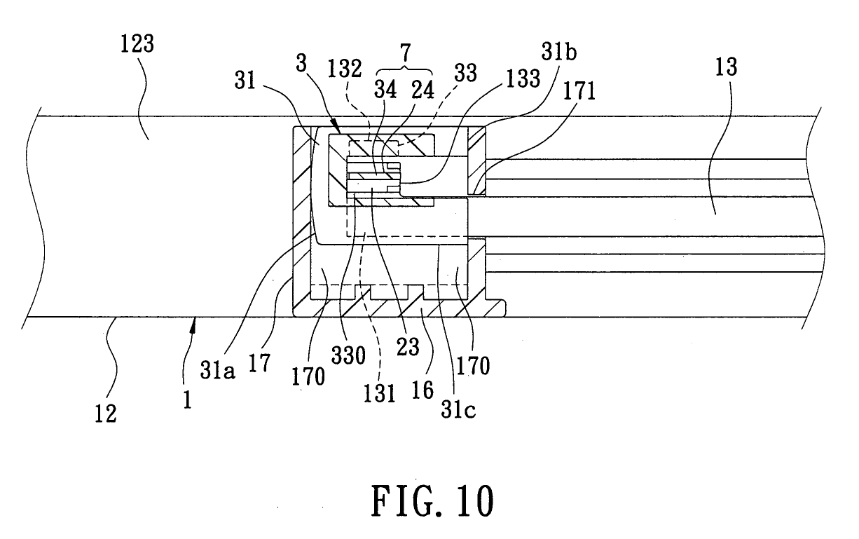

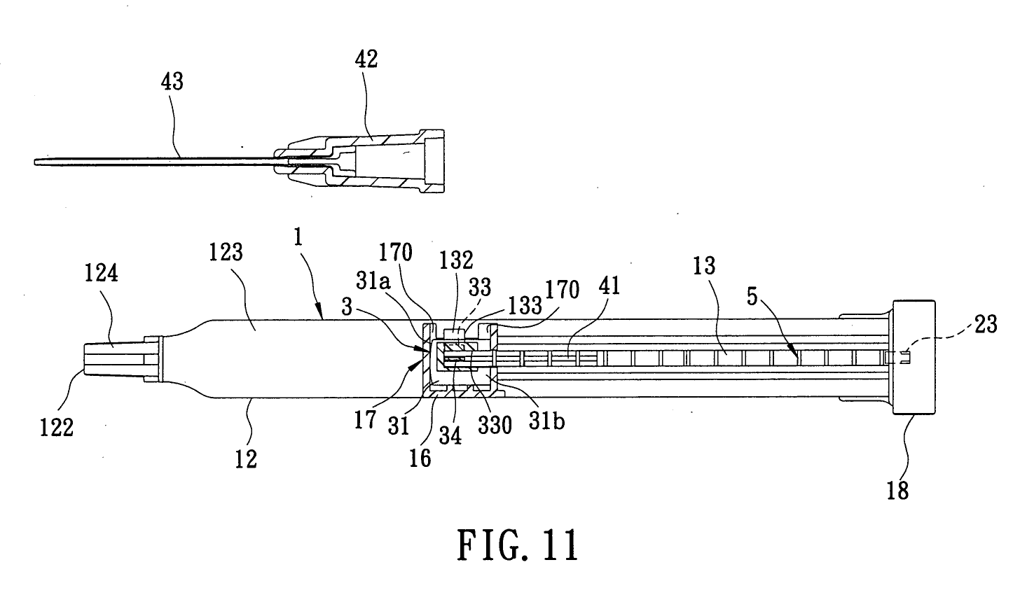

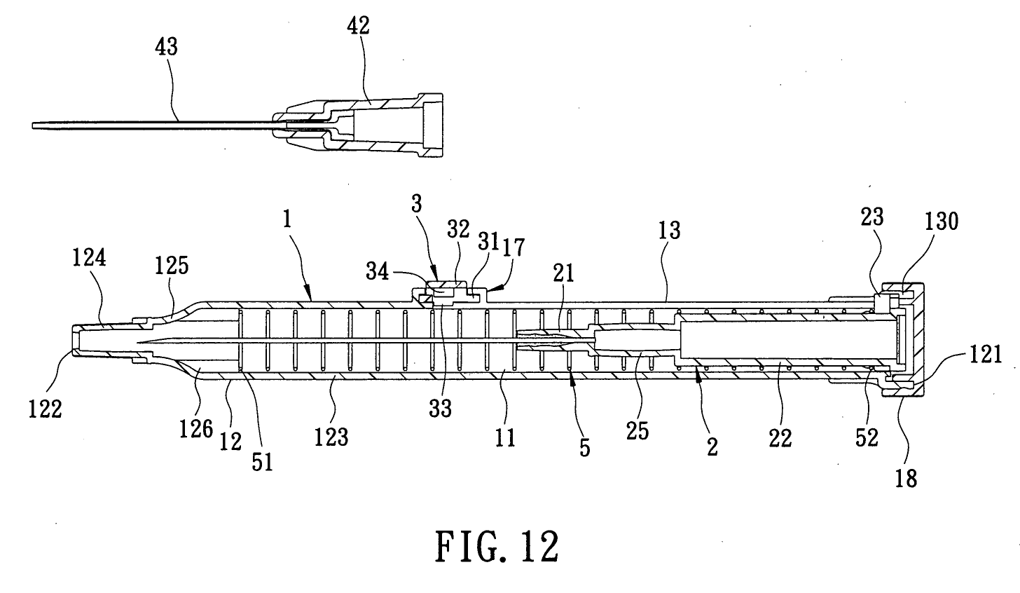

Referring to FIGS. 8 to 10, the second preferred embodiment of an intravenous catheter introducing device according to this invention is shown to be similar to the first embodiment in construction. The difference resides in that the pushing body 33 is disposed adjacent to the right wall surface 315, and extends from the inner major surface 312 towards the docking port 132. In this embodiment, the faceplate 32 is disposed on the outer major surface 311 of the guided body 31. The stabilizing unit 7 includes a retaining groove 24 which is formed in the stud end of the engaging peg 23 and which extends in the longitudinal direction, and a retaining pin 34 which is disposed adjacent to the left wall surface 314, and which extends from the ceiling surface 313 to be releasably retained in the retaining groove 24. Thus, by the engagement of the retaining pin 34 with the retaining groove 24, the engaging peg 23 is kept moving with the guided body 31 until the engaging peg 23 reaches the starting region 131, thereby ensuring steady movement of the engaging peg 23 between the docking shoulder 133 and the starting region 131. In addition, the retaining pin 34 is released from the retaining groove 24 once the engaging peg 23 reaches the starting region 131 so as to be thrust towards the ending region 130 by the biasing member 5, as shown in FIGS. 11 and 12.

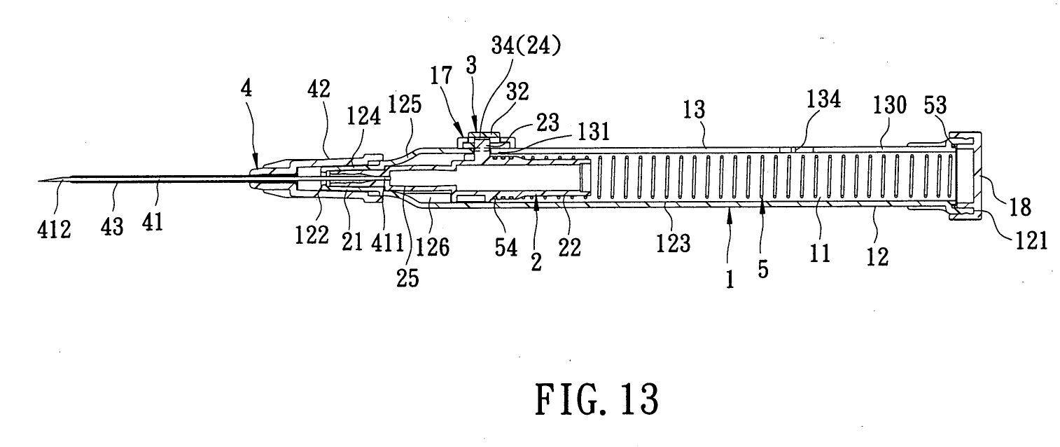

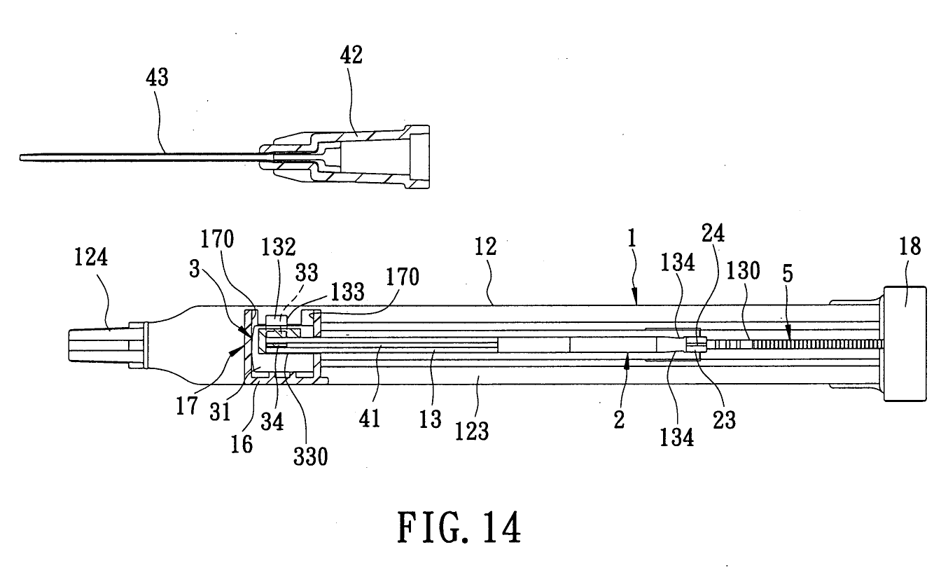

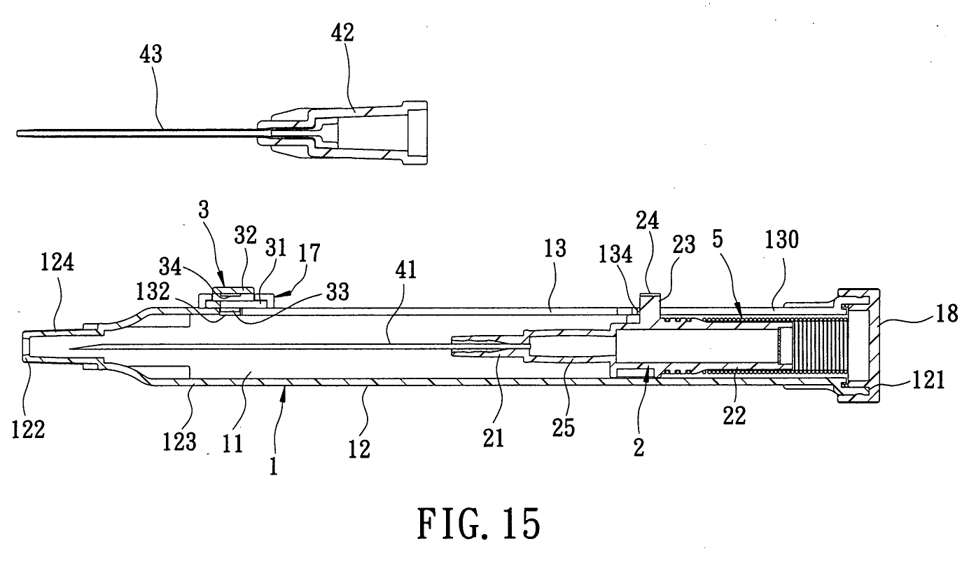

Referring to FIGS. 13 to 15, the third preferred embodiment of an intravenous catheter introducing device according to this invention is shown to be similar to the second embodiment in construction. In this embodiment, the biasing member 5 is a coil spring which is disposed in the rear wall portion 123, and which has a front biasing end 54 that engages the rear shell portion 22, and a rear biasing end 53 that engages the inner barrel wall surface adjacent to the rear open end 121 such that the biasing member 5 is in a tensed state when the needle hub 2 is in the position of use. In addition, the ending region 130 of the slotted path 13 in this embodiment is more elongated than that of the second embodiment, and a pair of retaining protrusions 134 are disposed in the ending region 130 such that the engaging peg 23, by moving over the retaining protrusions 134, is retained in the ending region 130, thereby preventing forward movement of the needle hub 2.

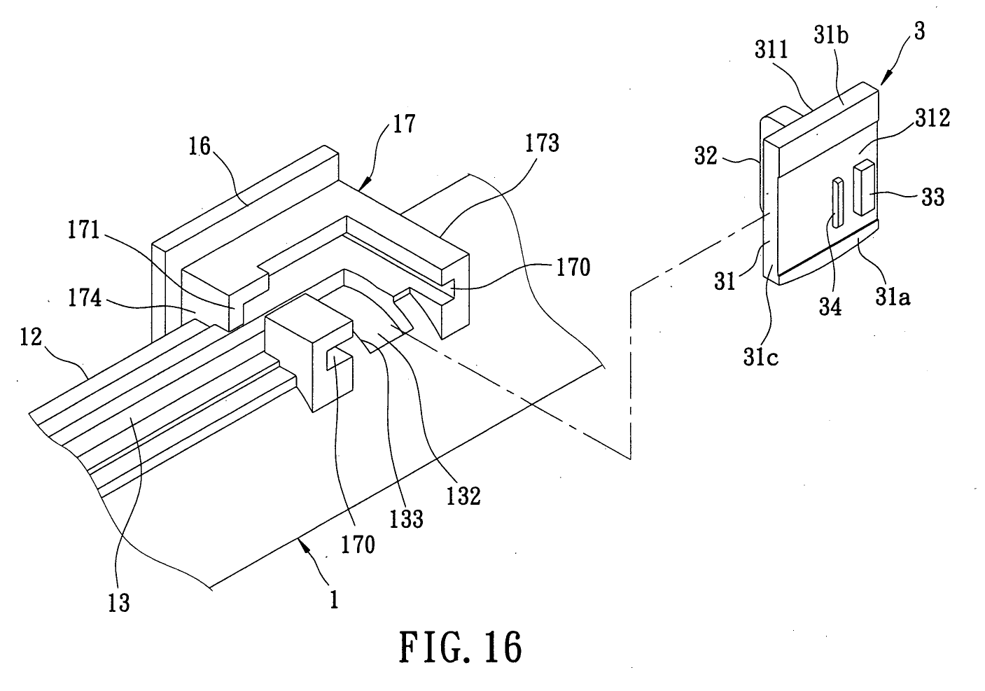

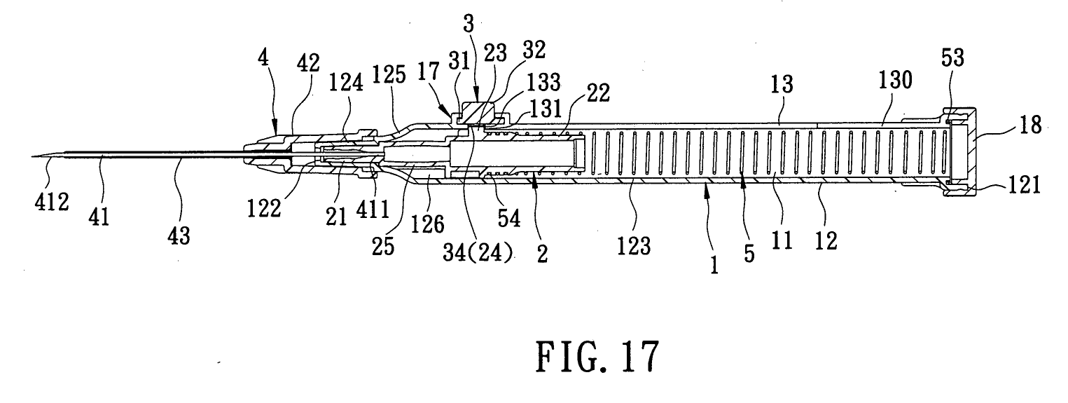

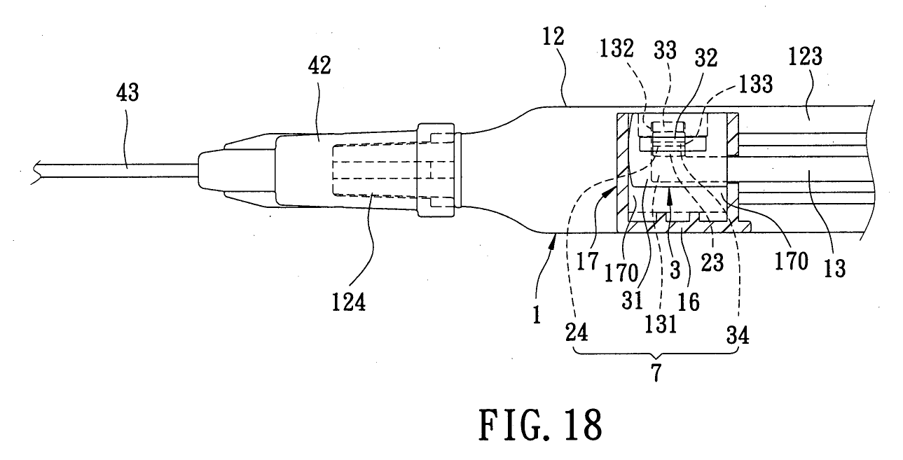

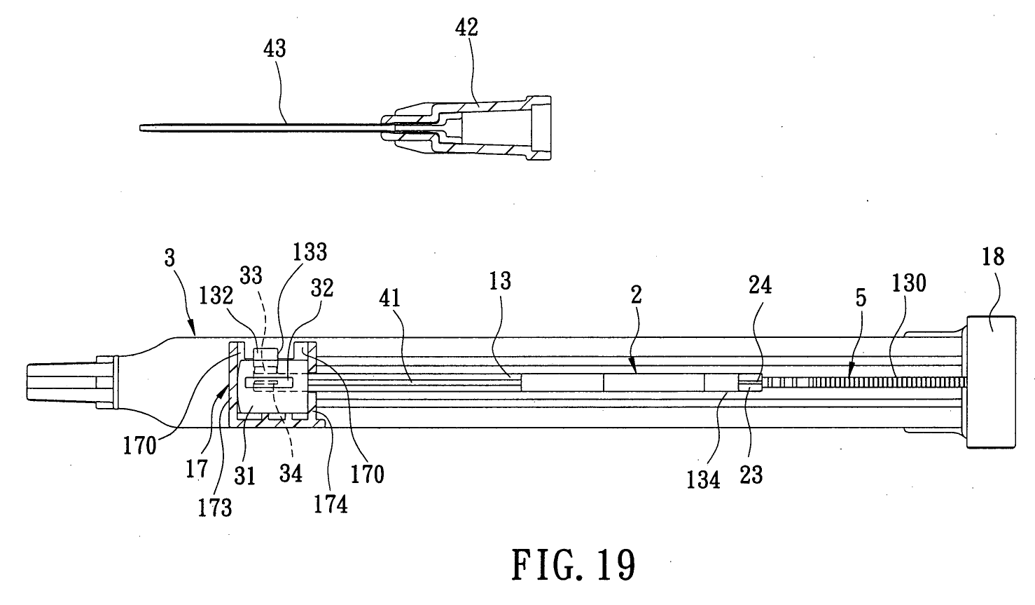

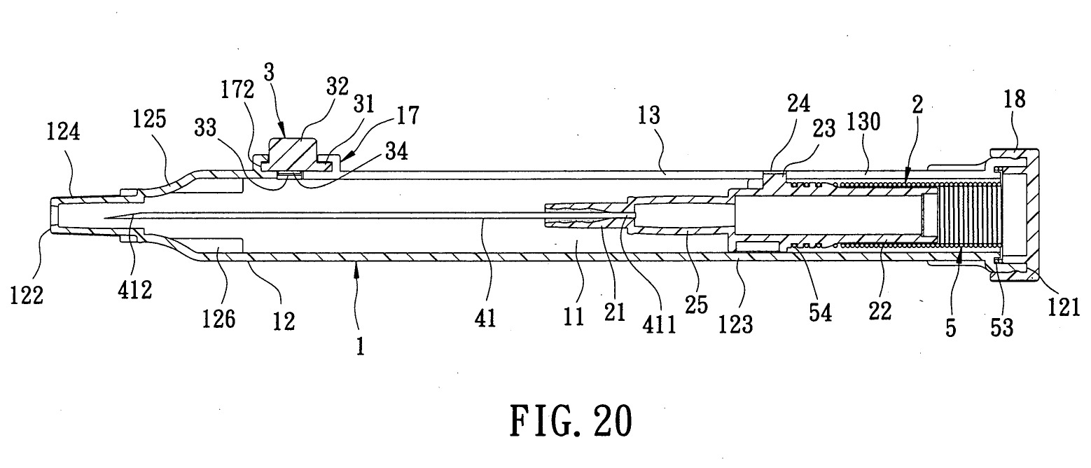

Referring to FIGS. 16 to 20, the fourth preferred embodiment of an intravenous catheter introducing device according to this invention is shown to be similar to the second embodiment in construction. In this embodiment, the guided body 31 of the actuating unit 3 is a flat plate without the cavity, and the stabilizing unit 7 includes a retaining groove 24 which is formed in the stud end of the engaging peg 23 and which extends in the longitudinal direction, and a retaining pin 34 which is disposed on the inner major surface 312 adjacent to the pushing body 33, and which is releasably retained in the retaining groove 24. Moreover, the biasing member 5, like that of the third embodiment, is a coil spring which is disposed in a tensed state when the needle hub 2 is in the position of use. In this embodiment, as the engaging peg 23 is slidable in the slotted path 13 and is disposed under the outer barrel wall surface in the ending region 130, as shown in FIG. 20, forward movement thereof can be prevented for inhibiting reuse.

To operate the engaging peg 23 for needle retraction, the user can hold the barrel 1 with one hand and operate the faceplate 32 with the same hand to cause the needle hub 2 to move to the disposal position for drawing the used needle cannula 41 into the passage 11. Therefore, the actuating operation is controllable by the user and is convenient and easy to conduct, and inadvertent needle-stick accidents can also be avoided. Moreover, the actuating unit 3 is compact in size for easy attachment to the barrel 1 so as to enable the device to have a compact and neat appearance.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

We claim:

1. An intravenous catheter introducing device comprising:
a barrel having front and rear open ends opposite to each other in a longitudinal direction, and a surrounding barrel wall which interconnects said front and rear open ends, said surrounding barrel wall including front and rear wall portions which are disposed proximate to said front and rear open ends, respectively, said rear wall portion having an inner barrel wall surface which surrounds an axis in the longitudinal direction and which defines a passage, and an outer barrel wall surface opposite to said inner barrel wall surface in radial directions relative to the axis, said inner barrel wall surface having a slotted path which extends through said outer barrel wall surface, which extends along the longitudinal direction, and which has starting and ending regions that are opposite to each other, and that are respectively distal from and proximate to said rear open end, said inner barrel wall surface further having a docking port which extends through said outer barrel wall surface, and which extends in a first transverse direction to be communicated with said starting region so as to form a docking shoulder that faces towards said front open end;
a needle cannula having a front segment terminating at a tip end, and a rear connecting end opposite to said front segment along the axis;
a needle hub including a front holding portion which is surrounded by said front wall portion and which holds said rear connecting end of said needle cannula, and a rear shell portion which is disposed opposite to said front holding portion along the axis to be inserted into said passage such that said needle hub is slidable relative to said surrounding barrel wall along the axis between a position of use, where said front segment of said needle cannula extends forwardly of said front open end, and a disposal position, where said front segment of said needle cannula is retracted into said passage, said needle hub further including an intermediate viewing-window portion which is interposed between said front holding portion and said rear shell portion;
a biasing member which is interposed between said rear shell portion and said inner barrel wall surface to bias said needle hub towards the disposal position;
an engaging peg which is configured to be anchored on said docking shoulder against the biasing action of said biasing member in the position of use, and which extends from said rear shell portion radially so as to be externally manipulated to move from said docking shoulder to said starting region, said engaging peg being dimensioned to be slidable along said slotted path to said ending region so as to move said needle hub to the disposal position;
a receptacle frame which is disposed on said outer barrel wall surface, said receptacle frame including front and rear guiding grooves which are spaced apart from each other in the longitudinal direction, and which cooperate with each other to define a guideway that extends over said docking port and said starting region in the first transverse direction;
an actuating unit including
a guided body which has first and second guided sides that are guided respectively in said front and rear guiding grooves when said actuating unit is moved along said guideway between normal and actuating positions, and
a pushing body which is configured such that, when said guided body is in the normal position, said pushing body is distant from said engaging peg, and when said guided body is in the actuating position, said pushing body shoves said engaging peg to said starting region; and
a stabilizing unit which is disposed between said guided body and said engaging peg, and which is configured such that said engaging peg is kept moving with said guided body until said engaging peg reaches said starting region, thereby ensuring steady movement of said engaging peg between said docking shoulder and said starting region.

2. The intravenous catheter introducing device according to claim 1, further comprising an end cap which is disposed to cover said rear open end, said ending region of said slotted path being in a constricted form and extending into said end cap such that said engaging peg is retained in said constricted ending region in the disposal position.

3. The intravenous catheter introducing device according to claim 1, wherein said engaging peg extends to terminate at a stud end which is disposed outwardly of said docking port so as to be shoved by said pushing body to said starting region.

4. The intravenous catheter introducing device according to claim 1, wherein said receptacle frame is integrally formed with said surrounding barrel wall, said front and rear guiding grooves respectively extending towards said front and rear open ends to terminate at front and rear guiding walls, respectively, said rear guiding wall being interrupted by said slotted path so as to permit said engaging peg to pass therethrough when said needle hub is moved from the position of use to the disposal position.

5. The intravenous catheter introducing device according to claim 1, wherein said guided body has a major wall which extends longitudinally to terminate at said first and second guided sides, and which has outer and inner major surfaces opposite to each other in a second transverse direction transverse to both the longitudinal direction and the first transverse direction, said stabilizing unit being disposed between said engaging peg and said inner major surface, said pushing body being disposed on said inner major surface and rightwardly of said engaging peg.

6. The intravenous catheter introducing device according to claim 5, wherein said inner major surface has a cavity which extends towards said outer major surface to terminate at a ceiling surface, and which extends in the first transverse direction to terminate at left and right wall surfaces that accommodate said engaging peg therebetween when said guided body is guided along said guideway, said left and right wall surfaces being disposed to extend rearwardly to form in said second guided side a port which is registered with said slotted path in the actuating position, thereby permitting said engaging peg to be moved to said ending region.

7. The intravenous catheter introducing device according to claim 6, wherein said pushing body is disposed on said right wall surface, said engaging peg having an enlarged head portion and a tail portion opposite to each other in the longitudinal direction, and left and right anchoring abutments which are interposed between said enlarged head portion and said tail portion, said right anchoring abutment being anchored on said docking shoulder when said needle hub is in the position of use, said stabilizing unit being disposed on said left wall surface and being formed with an auxiliary shoulder such that, in the normal position, said left anchoring abutment abuts against said auxiliary shoulder, and such that in the actuating position, said left anchoring abutment is disengaged from said auxiliary shoulder as a result of displacement of said auxiliary shoulder with said guided body to the actuating position.

8. The intravenous catheter introducing device according to claim 7, wherein said actuating unit further includes a faceplate which is disposed on said guided body distal from said cavity in the first transverse direction, which extends in the longitudinal direction, and which has front and rear fitted edges.

9. The intravenous catheter introducing device according to claim 8, wherein said receptacle frame includes front and rear jamb walls which are disposed rightwardly of said front and rear guiding grooves, respectively, and which are spaced apart from each other in the longitudinal direction to define a pushing path that is of such a dimension as to be fittingly engaged with said front and rear fitted edges of said faceplate.

10. The intravenous catheter introducing device according to claim 9, wherein said receptacle frame includes front and rear tiny projections which are disposed respectively on said front and rear jamb walls, and which are configured such that, once said faceplate is forced along the pushing path to reach the actuating position, said front and rear fitted edges of said faceplate are brought into a press-fit with said front and rear jamb walls by slipping over said front and rear tiny projections.

11. The intravenous catheter introducing device according to claim 7, wherein said receptacle frame includes a receiving groove which is disposed leftwardly of said slotted path, and which extends in the longitudinal direction to communicate said front guiding groove with said rear guiding groove, said receiving groove extending in the second transverse direction away from said slotted path to terminate at a shielding wall, said shielding wall extending rightwards to terminate at a lateral edge,
said major wall of said guided body having a leading edge which joins said front and rear guided sides, and which is to be received in said receiving groove, said outer major surface having lower and elevated regions respectively proximate to and distal from said leading edge, said elevated region being raised to accommodate contour of said ceiling surface so as to form a head-on surface which extends in the second transverse direction between said lower and elevated regions, and which is spaced apart from said lateral edge by a clearance when said actuating unit is in the normal position,
said intravenous catheter introducing device further comprising a safety guard which has an insert portion configured to be fittingly received in said clearance so as to guard against movement of said guided body away from the normal position.

12. The intravenous catheter introducing device according to claim 6, wherein said pushing body is disposed adjacent to said right wall surface, and extends from said inner major surface towards said docking port, said stabilizing unit including a retaining groove which is formed in said engaging peg and which extends in the longitudinal direction, and a retaining pin which is disposed adjacent to said left wall surface, and which extends from said ceiling surface to be releasably retained in said retaining groove such that said retaining pin is released from said retaining groove once said engaging peg reaches said starting region for being thrust towards said ending region by said biasing member.

13. The intravenous catheter introducing device according to claim 5, wherein said stabilizing unit includes a retaining groove which is formed in said engaging peg and which extends in the longitudinal direction, and a retaining pin which is disposed on said inner major surface adjacent to said pushing body, and which is releasably retained with said retaining groove such that said retaining pin is set free of said retaining groove once said engaging peg reaches said starting region so as to be thrust towards said ending region by said biasing member.

14. The intravenous catheter introducing device according to claim 1, wherein said biasing member is a coil spring which is disposed in said rear wall portion, which surrounds said rear shell portion, and which has a front biasing end that engages said inner barrel wall surface adjacent to said front wall portion, and a rear biasing end that engages said rear shell portion such that said biasing member is in a compressed state when said needle hub is in the position of use.

15. The intravenous catheter introducing device according to claim 1, wherein said biasing member is a coil spring which is disposed in said rear wall portion, and which has a front biasing end that engages said rear shell portion, and a rear biasing end that engages said inner barrel wall surface adjacent to said rear open end such that said biasing member is in a tensed state when said needle hub is in the position of use.

16. The intravenous catheter introducing device according to claim 1, wherein said rear wall portion further has a retaining protrusion which is disposed in said ending region of said slotted path such that said engaging peg, by moving over said retaining protrusion, is retained in said ending region, thereby preventing forward movement of said needle hub.
